(19) United States
(12) Patent Application Publication
(10) Pub. No.: US 2006/0004055 A1
(43) Pub. Date: Jan. 5, 2006

(54) Derivative of 5-(4-bromophenyl)-1-(2,4-dichlorophenyl)-4-ethyl-N-(piperidine-1-yl)-1H-pyrazole-3-carboxamide, the preparation and therapeutic use thereof

(75) Inventors: Gilles Miscoria, La Boissiere (FR); Murielle Rinaldi, Saint Georges D'Orques (FR); Joseph Schofield, Corbeil-Essonnes (FR)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Sanofi-Aventis, Paris (FR)

(21) Appl. No.: 11/159,779

(22) Filed: Jun. 23, 2005

(30) Foreign Application Priority Data
Dec. 23, 2002 (FR) 02/16688

(51) Int. Cl. A61K 31/454; C07D 43/02

(52) U.S. Cl. 514/326; 546/211

(57) ABSTRACT

The invention relates to a derivative of 5-(4-bromophenyl)-1-(2,4-dichlorophenyl)-4-ethyl-N-(piperidin-1-yl)-1H-pyrazole-3-carboxamide of general formula (I): 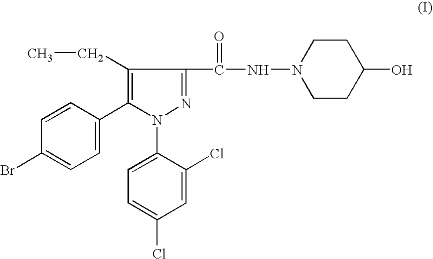 Preparation process and therapeutic use.

This application is a continuation of International Application No. PCT/FR2003/003814, filed Dec. 19, 2003, which claims the benefit of priority of French Application No. 02/16688 filed Dec. 23, 2002.

The present invention relates to a derivative of 5-(4-bromophenyl)-1-(2,4-dichlorophenyl)-4-ethyl-N-(piperidin-1-yl)-1H-pyrazole-3-carboxamide, to the preparation thereof and to the therapeutic use thereof.

5-(4-Bromophenyl)-1-(2,4-dichlorophenyl)-4-ethyl-N-(piperidin-1-yl)-1H-pyrazole-3-carboxamide is described in international patent application WO 00/46209. Moreover, derivatives of 1,5-diphenyl-1H-pyrazole-3-carboxamide are described in European patent EP-0 576 357.

A subject of the present invention is a compound corresponding to formula (I):

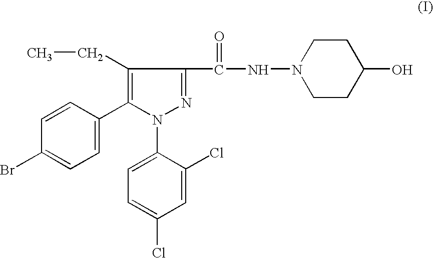

The compound of formula (I) can exist in the form of a salt. Such addition salts form part of the invention.

These salts are advantageously prepared with pharmaceutically acceptable acids, but the salts of other acids useful, for example, for purifying or isolating the compounds of formula (I) also form part of the invention.

The compound of formula (I) can also exist in the form of hydrates or of solvates, i.e. in the form of associations or of combinations with one or more molecules of water or with a solvent. Such hydrates and solvates also form part of the invention.

Thus, the compound of formula (I) that is the subject of the invention is 5-(4-bromophenyl)-1-(2,4-dichlorophenyl)-4-ethyl-N-(piperidin-1-yl)-1H-pyrazole-3-carboxamide.

In accordance with the invention, the compound of formula (I) can be prepared according to the process that follows. This process is characterized in that a functional derivative of 5-(4-bromophenyl)-1-(2,4-dichlorophenyl)-4-ethyl-1H-pyrazole-3-carboxylic acid of formula: 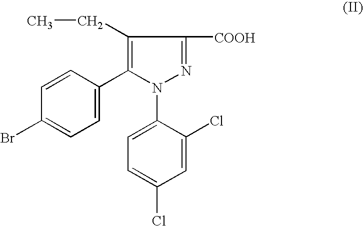
is treated with a derivative of 1-aminopiperidine of formula:

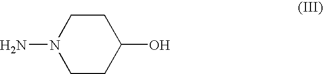

The reaction is carried out in a basic medium, for example in the presence of triethylamine in an inert solvent such as dichloromethane or tetrahydrofuran.

As a functional derivative of the acid (II), use may be made of the acid chloride, the anhydride, a mixed anhydride, a C₁-C₄ alkyl ester in which the alkyl is straight or branched, an activated ester, for example p-nitrophenyl ester, or the free acid opportunistically activated, for example, with N,N-dicyclohexylcarbodiimide or with benzotriazole-N-oxotris(dimethylamino)phosphonium hexafluorophosphate (BOP).

Thus, by means of the process according to the invention, it is possible to react the chloride with the acid of formula (II) obtained by reaction of thionyl chloride with the acid of formula (II) in an inert solvent, such as benzene or toluene, or a chlorinated solvent (dichloromethane, dichloroethane or chloroform, for example), an ether (tetrahydrofuran or dioxane, for example) or an amide (N,N-dimethylformamide, for example), in an inert atmosphere, at a temperature of between 0° C. and the reflux temperature of the solvent.

The compound of formula (II) is prepared according to patent application WO 00/46209.

The (4-hydroxy)-N-aminopiperidine of formula (III) is prepared according to the reaction scheme below:

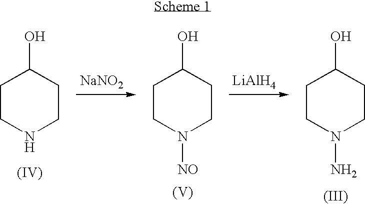

The nitrosamine derivative of formula (V) is prepared from 4-hydroxypiperidine by reacting sodium nitrite in water.

The reduction of the nitrosamine derivative of formula (V) is carried out in the presence of lithium aluminum hydride in an anhydrous solvent such as tetrahydrofuran (THF).

The following example describes the preparation of the compound in accordance with the invention.

This example is not limiting and merely illustrates the present invention.

In the example, the following abbreviations are used:
EtOAc: ethyl acetate
THF: tetrahydrofuran.

For the proton Nuclear Magnetic Resonance (NMR) spectra measured at 200 MHz in DMSO-d₆, the chemical shifts observed are expressed in the following way: s: singlet; bs: broad singlet; d: doublet; t: triplet; m: multiplet; bm: broad multiplet.

### EXAMPLE 1

### A: 1-Nitrosopiperidin-4-ol

15 g of piperidin-4-ol are dissolved in 65 ml of water and the solution is cooled to between 0° C. and 5° C. by means of an ice bath, and then the solution formed is run dropwise into a solution containing 20.5 g of sodium nitrite in 65 ml of water, maintaining the temperature at less than 5° C. 12 ml of acetic acid are added and then the mixture is left to return to ambient temperature and left overnight with stirring. It is cooled in an ice bath and solid Na₂CO₃ is added so as to reach a pH of greater than 7. Water is added, and the mixture is extracted with EtOAc, separated by settling out, then dried over MgSO₄ and concentrated to dryness under vacuum. The expected compound is obtained in the form of an oil, m=16.11 g.

### B: 1-Aminopiperidin-4-ol

5 g of LiAlH₄ are suspended in 70 ml of anhydrous THF, under nitrogen, the suspension is cooled to between 0° C. and 5° C. and 10% of a solution containing 8 g of 1-nitrosopiperidin-4-ol in 40 ml of anhydrous THF is run in dropwise, the temperature is controlled by means of an ice bath, and 50 ml of THF are added followed by the remaining 1-nitrosopiperidin-4-ol solution. The reaction medium is refluxed for 3 hours and then left overnight at ambient temperature. It is cooled to between 0° C. and 5° C. in an ice bath, and then 5 ml of water, followed by 5 ml of a 15% NaOH solution and a further 15 ml of water are added slowly. After stirring at ambient temperature for one hour, the reaction medium is filtered, thorough rinsing is performed with THF, and the product is concentrated under vacuum. The oil obtained is chromatographed on alumina, elution being carried out with a CHCl₃/MeOH mixture (96/4; v/v). The expected compound is obtained in the form of an oil, m=2.5 g.

NMR DMSO 4.4 ppm: bs: 1H; 3.3 ppm: m: 1H; 2.6 and 2.0 ppm: bm: 4H; 1.6 and 1.3 ppm: bm: 4H.

### C: 5-(4-Bromophenyl)-1-(2,4-dichlorophenyl)-4-ethyl-N-(hydroxypiperidin-1-yl)-1H-pyrazole-3-carboxamide

1.46 g of 1-aminopiperidin-4-ol are added to 100 ml of CH₂Cl₂, under nitrogen, 3.18 ml of triethylamine are added, and then a solution containing 5.26 g of acid chloride of 5-(4-bromophenyl)-1-(2,4-dichlorophenyl)-4-ethyl-N-(4-hydroxypiperidin-1-yl)-1H-pyrazole-3-carboxylic acid in 50 ml of CH₂Cl₂ is run in, at a temperature of between 0° C. and 5° C. The mixture is left at 4° C. overnight, and the reaction medium is then poured onto ice-cold water and separated by settling out. The organic phase is washed with a 5% Na₂CO₃ solution and a saturated NaCl solution, and then dried over MgSO₄ and concentrated to dryness under vacuum. The residue obtained is purified by chromatography on silica, elution being carried out with a toluene/ethyl acetate mixture (80/20; v/v). After elimination of the solvent, 3.7 g of the expected product are obtained, which product crystallizes from isopropyl ether, M.p.=178° C.

The compound according to the invention was the subject of pharmacological assays for determining its cannabinoid CB₁ receptor antagonist action.

The compound of formula (I) has very good affinity, in vitro (IC₅₀=32 nM), for cannabinoid CB₁ receptors, under the experimental conditions described by M. Rinaldi-Carmona et al. (FEBS Letters, 1994, 350, 240-244).

The antagonist nature of the compound of formula (I) was demonstrated by the results obtained in the models of adenylate cyclase inhibition as described in M. Rinaldi-Carmona et al., J. Pharmacol. Exp. Ther., 1996, 278, 871-878.

The toxicity of the compound of formula (I) is compatible with its use as a medicinal product.

According to another of its aspects, the subject of the present invention is medicinal products that comprise a compound of formula (I), or one of its pharmaceutically acceptable salts, solvates or hydrates. The medicinal products may be useful for preventing or treating diseases involving cannabinoid CB₁ receptors.

For example and in a nonlimiting manner, the compound of formula (I) is useful as a psychotropic medicinal product, in particular for the treatment of psychiatric disorders including anxiety, depression, mood disorders, insomnia, deliria disorders, obsessive disorders, psychoses in general and schizophrenia, and also for the treatment of disorders associated with the use of psychotropic substances, in particular in the case of substance abuse and/or substance dependency, including alcohol dependency and nicotine dependency.

The compound of formula (I) according to the invention may be used as a medicinal product for the treatment of migraine, stress, diseases of psychosomatic origin, panic attacks, epilepsy, movement disorders, in particular dyskinesia or Parkinson's disease, trembling and dystonia.

The compound of formula (I) according to the invention may also be used as a medicinal product in the treatment of memory disorders, cognitive disorders, in particular in the treatment of senile dementia and Alzheimer's disease, and also in the treatment of attention or alertness disorders. In addition, the compound of formula (I) may be useful as a neuroprotective agent, in the treatment of ischemia, cranial traumas and the treatment of neurodegenerative diseases: including chorea, Huntington's chorea and Tourrette's syndrome.

The compound of formula (I) according to the invention may be used as a medicinal product in the treatment of pain: neuropathic pain, acute peripheral pain and chronic pain of inflammatory origin.

The compound of formula (I) according to the invention may be used as a medicinal product in the treatment of appetite disorders, craving disorders (craving for sugars, carbohydrates, drugs, alcohol or any appetizing substance) and/or eating disorders, in particular as an anorexigenic agent or for the treatment of obesity or of bulimia, and also for the treatment of type II diabetes or non-insulin-dependent diabetes. Furthermore, the compound of formula (I) according to the invention may be used as a medicinal product in the treatment of gastrointestinal disorders, diarrheic disorders, ulcers, vomiting, bladder and urinary disorders, disorders of endocrine origin, cardiovascular disorders, hypotension, hemorrhagic shock, septic shock, chronic liver cirrhosis, asthma, Raynaud's syndrome, glaucoma, fertility disorders, inflammatory phenomena, immune system diseases, in particular autoimmune and neuroinflammatory diseases such as rheumatoid arthritis, reactional arthritis, diseases resulting in demyelinization, multiple sclerosis, infectious and viral diseases such as encephalitis, cerebral strokes, and also as medicinal products for anticancer chemotherapy and for the treatment of Guillain-Barré syndrome.

According to the present invention, the compound of formula (I) is most particularly useful for the treatment of psychotic disorders, in particular schizophrenia; for the treatment of appetite disorders and obesity; for the treatment of memory disorders and cognitive disorders; for the treatment of alcohol dependency and nicotine dependency, i.e. for alcohol withdrawal and for tobacco withdrawal.

According to another of its aspects, the present invention relates to pharmaceutical compositions comprising, as active principle, a compound according to the invention. These pharmaceutical compositions contain an effective dose of the compound according to the invention, or a pharmaceutically acceptable salt, a hydrate or a solvate of said compound, and also at least one pharmaceutically acceptable excipient.

Said excipients are chosen, according to the pharmaceutical form and the method of administration desired, from the usual excipients that are known to those skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or its optional salt, solvate or hydrate, can be administered in a unit form of administration, as a mixture with conventional pharmaceutical excipients, to animals and to human beings for the prophylaxis or the treatment of the disorders or the diseases above.

The appropriate unit forms of administration include oral forms such as tablets, soft or hard gelatin capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms and implants. For topical application, the compounds according to the invention may be used in creams, gels, ointments or lotions.

When given orally, the dose of active principle administered per day may reach 0.01 to 100 mg/kg, taken in one or more doses, preferably 0.02 to 50 mg/kg.

There may be specific cases where higher or lower dosages are appropriate; such dosages do not depart from the context of the invention. According to the usual practice, the dosage appropriate for each patient is determined by the physician according to the method of administration, and the weight and the response of said patient.

According to another of its aspects, the present invention also relates to a method of treating the pathologies indicated above, which comprises the administration, to a patient, of an effective dose of a compound according to the invention, or one of its pharmaceutically acceptable salts or hydrates or solvates.

1. A compound of formula (I): 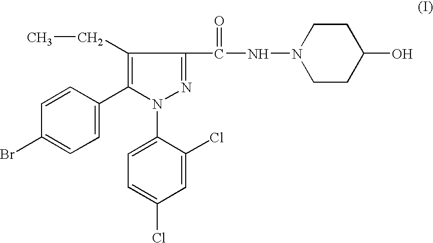
or a salt, hydrate or solvate thereof.

2. A process for preparing a compound of formula (I) as claimed in claim 1, comprising reacting 5-(4-bromophenyl)-1-(2,4-dichlorophenyl)-4-ethyl-N-(piperidin-1-yl)-1H-pyrazole-3-carboxylic acid of formula (II): 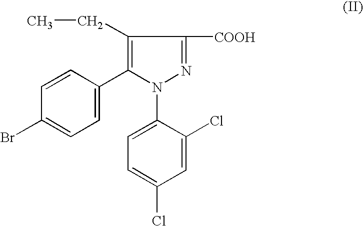
with 1-aminopiperidine of formula (III):

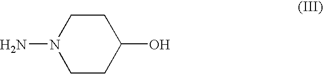

3. A pharmaceutical composition, comprising a compound according to claim 1, or a pharmaceutically acceptable salt, hydrate or solvate thereof, and at least one pharmaceutically acceptable excipient.

4. A method of treating diseases involving the cannabinoid CB₁ receptor comprising administering to a patient in need of such treatment an effective amount of a compound according to claim 1.

5. The method according to claim 4, wherein the disease is appetite disorders or obesity.

6. The method according to claim 4, wherein the disease is memory disorders or cognitive disorders.

7. The method according to claim 4, wherein the disease is alcohol dependency or nicotine dependency.
